(19) United States
(12) Patent Application Publication
(10) Pub. No.: US 2012/0157551 A1
(43) Pub. Date: Jun. 21, 2012

(54) Use of Polymers

(75) Inventors: Paul Hugh Findlay, Liverpool (GB); Brodyck James Lachlan Royles, Liverpool (GB); Roselyne Marie Andree Baudry, Liverpool (GB); Neil John Simpson, Liverpool (GB); Sharon Todd, Liverpool (GB); Steven Paul Rannard, Liverpool (GB)

(73) Assignee: UNILEVER PLC, London (GB)

(21) Appl. No.: 13/394,092

(22) PCT Filed: Sep. 8, 2010
(86) PCT No.: PCT/EP2010/005501

§ 371 Date: Mar. 2, 2012

(30) Foreign Application Priority Data
Sep. 8, 2009 (GB) 0915682.9

(51) Int. Cl. A61K 47/32; C08L 47/00; C09D 147/00; C09K 5/00; C09D 11/00; C10M 145/14; C09K 8/60; G03F 7/004; A61K 8/81; C09J 147/00

(52) U.S. Cl. 514/772.6; 525/55; 524/502; 508/469; 507/224; 430/270.1; 252/67

(57) ABSTRACT

The present invention relates to the use of branched addition copolymers possessing melt or solution viscosities lower than the linear polymer analogues of equivalent or greater weight average molecular weight, compositions comprising said copolymers, methods for their preparation, and he use of novel polymers per se in for example but not limited to solution formulations or melt formulations.

### TECHNICAL FIELD

The present invention relates to the use of branched addition copolymers possessing melt or solution viscosities lower than the linear polymer analogues of equivalent or greater weight average molecular weight, compositions comprising said copolymers, methods for their preparation, and the use of novel branched addition copolymers per se in for example but not limited to solution or melt formulations.

More specifically the present invention relates to the use of branched addition copolymers as a replacement for linear polymer analogues of equivalent or greater weight average molecular weight, such that the viscosities of the melt or solution are lower than the viscosity values for linear polymer analogues of equivalent or greater weight average molecular weight, compositions comprising said copolymers, methods for their preparation, and the use of novel branched addition copolymers per se in for example but not limited to solution or melt formulations.

In addition the present invention relates to the use of branched addition copolymers as a replacement for linear polymer analogues of equivalent or greater weight average molecular weight to reduce the elastic behaviour of a polymeric solution or melt formulation, when compared with the values obtained for a linear polymeric material when used alone.

### BACKGROUND OF THE INVENTION

### Polymer Rheology

Viscosity is defined as the resistance of a fluid to deformation under an external stress. The viscosity of a solution is governed by the internal structure of the pure liquid or by the nature of the material dissolved or dispersed in the liquid phase. Polymeric materials dissolved in a solvent or alternatively in a molten form, typically demonstrate high viscosity values. In many cases this is advantageous to a system in which the polymeric material is placed and indeed it is commonplace for a polymer to be designed to act in just such a way and to cause an increase in viscosity, such as in the use of polymeric thickeners in a wide range of applications.

In several applications however, this high solution or melt viscosity is not desirable as it renders the formulation intractable or at the very least difficult to process or utilise in final form. In such situations it is necessary for the formulation to be either diluted with further solvent or used at a higher temperature to give a formulation of the required viscosity. An example of such a situation is in the field of coatings where the viscosity of the final coating formulation is crucial for efficient application and coverage of the coating onto the substrate. In such applications, a large amount of solvent is often required to give a workable solution. Unfortunately, where the solvent in question is a volatile organic compound (VOC) the use thereof can lead to encroachment upon environmental legislation.

In melt processing the high viscosity of high molecular weight polymers can lead to processing difficulties with the result that high temperatures are required. The use of high temperatures in processing thus leads to high energy requirements. In many applications however, polymers are required to possess high molecular weights in order to give suitable final properties, with the result that the polymers give rise to extremely high solution or melt viscosities, which as discussed above can be problematic.

Polymeric solutions or melts also exhibit elastic behaviour and deformation under external stress. That is, polymers in a solution or a melt exhibit chain entanglement which manifests itself in an elastic behaviour of the polymer solution or melt. This effect is particularly noticeable in high molecular weight linear systems where the increased levels of chain entanglements often results in the creation of highly elastic solutions or melts. In many applications this is not advantageous as it can render the melt or solution intractable and difficult to process. This elastic or “stringiness” in a formulation can also limit the amount of polymer that can be incorporated. In many areas the elastic behaviour of a formulation can affect the mode of application this is particularly true when spraying, injecting, jetting or roller applying the formulation.

It has now surprisingly been found that branched addition copolymeric structures do not exhibit this effect to such an extent as linear materials of equivalent molecular weight. Therefore also in accordance with the present invention there is described the use of branched addition copolymers as a replacement for linear analogues to reduce the elastic behaviour, of a melt or solution compared to the elastic characteristics of a linear polymeric material when used alone.

By the term ‘equivalent analogous linear polymer’ is meant a polymer of identical or similar chemical composition or molecular weight. For example a branched copolymer containing seventy weight percent styrene component and a linear polymer containing a seventy percent styrene component of equivalent molecular weight.

The term ‘melt’ and ‘solution’ are used herein to describe that the polymer relates to a formulation where the polymer is molten or softened via heat or is dissolved in a suitable solvent respectively.

Whilst not wishing to be bound by any particular theory, it is thought that polymer viscosity is primarily due to the entanglement of the polymer chains in a system. Where the molecular weight or chain length reaches a critical point molecular weight, M_{c}, there is a sharp increase in viscosity. In addition, where the polymers posses inter or intra-molecular associating moieties, such as H-bond donor or accepting units, the M_{c} can be quite small leading to extremely viscous solutions or melts, such as in the case of many natural or functionalised polysaccharides or synthetic water-soluble macromolecules.

Linear polymers are commonly used in many applications due to their high solubility and ease of preparation. However, due to the architecture of these copolymers, the copolymers often give rise to high viscosity solutions or melts. In addition such linear polymers can be extremely slow or difficult to dissolve or melt in order to achieve isotropic liquids.

There has been a considerable amount of recent work undertaken to try and address the problematic issues alluded to above with the result that it has now been found that dendritic polymers possess lower solution and melt viscosities due to the more globular architecture of such molecules. Dendrimers in particular have been shown to give low solution and melt viscosities and due to the perfect nature of their structures typically do not show a M_{c}. Indeed in many cases dendrimers show a decrease in solution or melt viscosity above a particular molecular weight. The synthesis of dendritic materials however is extremely tedious typically requiring a multi-step synthetic route where the ultimate molecular weights or chemical functionalities are limited. For these reasons dendrimers are extremely expensive to prepare when compared to commercially available polymers and are therefore only suitable for a limited number of high end applications.

### Branched Polymers

Branched polymers are polymer molecules of a finite size which are branched. Branched polymers differ from cross-linked polymer networks which tend towards an infinite size having interconnected molecules and which are generally not soluble. In some instances, branched polymers have advantageous properties when compared to analogous linear polymers. For example, higher molecular weights of branched copolymers can be solubilised more easily than those of corresponding linear polymers. In addition, as branched polymers tend to have more end groups than linear polymers they generally exhibit strong surface-modification properties. Branched polymers are therefore useful components of many compositions and their utilisation in a variety of applications is desirable.

The inventors have now found that branched polymers also possess lower solution or melt viscosities presumably due to their non-linear architecture. In addition, unlike dendrimers, such compounds typically show non-ideal branching in structure and can possess polydisperse structures and molecular weights. Furthermore, the preparation of branched polymers is much more readily achieved than their dendrimer counterparts and although the ultimate structure of such polymers is neither perfect nor mono-disperse, branched (or hyperbranched) polymers are far more suitable and economical to produce for a variety of industrial applications.

Branched polymers are usually prepared via a step-growth mechanism via the polycondensation of suitable monomers and are usually limited by the choice of monomers, chemical functionality of the resulting polymer and the molecular weight. In addition polymerisation, a one-step process may be employed in which a multifunctional monomer is used to provide functionality in the polymer chain.

However, a limitation on the use of a conventional one-step process is that the amount of multifunctional monomer must be carefully controlled, usually to substantially less than 0.5% w/w in order to avoid extensive cross-linking of the polymer and the formation of insoluble gels. It is difficult to avoid cross-linking using this method, especially in the absence of a solvent as a diluent and/or at high conversion of monomer to polymer.

WO 99/46301 discloses a method of preparing a branched polymer comprising the steps of mixing together a monofunctional vinylic monomer with from 0.3 to 100% w/w (of the weight of the monofunctional monomer) of a multifunctional vinylic monomer and from 0.0001 to 50% w/w (of the weight of the monofunctional monomer) of a chain transfer agent and optionally a free-radical polymerisation initiator and thereafter reacting said mixture to form a copolymer. The examples of WO 99/46301 describe the preparation of primarily hydrophobic polymers and, in particular, polymers wherein methyl methacrylate constitutes the monofunctional monomer. These polymers are useful as components in reducing the melt viscosity of linear poly(methyl methacrylate) in the production of moulding resins.

WO 99/46310 discloses a method of preparing a (meth)acrylate functionalised polymer comprising the steps of mixing together a monofunctional vinylic monomer with from 0.3 to 100% w/w (based on monofunctional monomer) of a polyfunctional vinylic monomer and from 0.0001 to 50% w/w of a chain transfer agent, reacting said mixture to form a polymer and terminating the polymerisation reaction before 99 % conversion. The resulting polymers are useful as components of surface coatings and inks, as moulding resins or in curable compounds, for example curable moulding resins or photoresists.

WO 02/34793 discloses a rheology modifying copolymer composition containing a branched copolymer of an unsaturated carboxylic acid, a hydrophobic monomer, a hydrophobic chain transfer agent, a cross-linking agent, and, optionally, a steric stabilizer. The copolymer provides increased viscosity in aqueous electrolyte-containing environments at elevated pH. The method for production is a solution polymerisation process. The polymer is lightly cross-linked, less than 0.25%.

U.S. Pat. No. 6,020,291 discloses aqueous metal working fluids used as lubricants in metal cutting operations. The fluids contain a mist-suppressing branched copolymer, including hydrophobic and hydrophilic monomers, and optionally a monomer comprising two or more ethylenically unsaturated bonds. Optionally, the metal working fluid may be an oil-in-water emulsion. The polymers are based on poly(acrylamides) comprising sulfonate-containing and hydrophobically modified monomers. The polymers are cross-linked to a very small extent by using very low amounts of bis-acrylamide, without using a chain transfer agent.

Hawker et al (Journal of the American Chemical Society, 1995, 117, 4409) describe the use of a family of dendritic polyether macromolecules which give rise to reduced melt viscosities and which do no possess a M_{c}. The authors of this paper describe this effect as being like ‘molecular ball-bearings’. The dendrimers are made via a multi-step convergent growth approach where they show molecular weights up to 20,993 Da.

Hawker et al (Material Research and Innovation 2002, 6, 160) describes the melt viscosity of polybenzyl ether dendrimers and polybenzyl ether dendrimers to which a polystyrene unit has been covalently attached. In both cases the melt viscosity of these materials was significantly lower than the equivalent linear polystyrene of equivalent molecular weight. In addition, the pure dendrimers show Newtonian behaviour supporting the hypothesis that branching greatly reduces the chain entanglement of polymeric melts

Matyjaszewski et al (Macromolecules, 1996, 29, 1079) describes the synthesis of branched styrenic polymers via the homopolymerisation of chloromethyl styrene via an atom transfer radical polymerisation (ATRP). The intrinsic viscosities (as determined from their size exclusion chromatograms) of these polymers were found to be lower than linear polystyrene of equivalent weight average molecular weight.

US 2007/0208143 describes the synthesis of a water-soluble branched polymer having high molecular weight and low solution viscosity prepared in a step-wise manner whereby the polymer has a composition obtained from 60 to 99.5 mol % (meth)acrylamide, 0.5 to 20 mol % of an α,β-unsaturated carboxylic acid monomer, 1 to 20 mol % of a water-soluble cationic monomer, 0.01 to 1 mol % of a chain transfer agent and 0.005 to 5 mol % of a cross-linkable monomer. The polymerisation is via a two step process in an aqueous solvent whereby the first step constitutes the addition of a persufate initiator to form a pre-polymer followed by addition of a second aliquot of initiator and monomer followed by termination of the polymerisation at the required solution viscosity. The polymers show reduced solution viscosities and are applicable to papermaking.

U.S. Pat. No. 6,262,223 describes a method for the production of composite structures formed via the incorporation of aromatic triamines in the preparation of branched polyimides. These star-branched materials show lower melt viscosities due to their molecular architectures. The polymers are particularly suitable for polymer matrix composites.

Dvornic et al (Macromolecules, 1998, 31, 4498) describes the solution behaviour of varying generations of ethylenediamine—core polyamidoamine (PAMAM) dendrimers. Herein it is described that the solution behaviour of dendrimers with molecular weights ranging from around 500 to 60,000 Da was investigated. It was found that solutions of the dendrimers exhibited Newtonian behaviour and showed a reduced zero shear viscosity when compared to a linear PAMAM polymer of equivalent molecular weight. In addition, the dendrimers studied did not show a critical molecular weight for entanglement M_{c}.

WO 2008/071662 (Unilever) describes the preparation of branched addition polymers wherein a component of the polymer, monofunctional monomer, polyfunctional monomer or chain transfer agent, has a molecular weight of at least 1000 Daltons. The polymers are described as aiding the colloid stabilisation in laundry formulations.

U.S. Pat. No. 5,777,054 Amcol describes the preparation and use of a cross-linked porous polymer microparticle prepared using mono and polyunsaturated monomers via a suspension route. The polymers are wholly cross-linked and are therefore insoluble and are used in oil separation processes.

Russian Chemical Bulletin (2007) 56(2). 197-204 describes the photocromic transformations of 6-nitrospyrans in polymer matrices including branched polymers based on methyl methacrylate. The quenching of the absorbance is effected by the polymer architecture with differences being observed between linear and branched polymers. The viscosity profiles of the polymer-dye adducts is not discussed.

### DETAILED DESCRIPTION

Polymers are ubiquitous in their everyday usage. A common application of these materials is as viscosity modifiers in solution where they essentially thicken many formulations ranging from shower gels to topical pharmaceutical products. In these applications the intrinsic high molecular weight of the polymers, molecular association, chain entanglement and ultimate rise in the formulation viscosity is advantageous. In many applications however, a low viscosity formulation is desirable and many different routes have been used to achieve this including: increasing the amount of solvent, the addition of a low molecular weight viscosity reducer, the use of a “reactive diluent” or by heating the formulation. In applications such as in coatings, lubricants or adhesives the reduction in solvent while maintaining an equivalent solution viscosity is particularly attractive. Here the polymer usually imparts key benefits such as film-formation, curing and adhesion or friction reduction. The reduction in volatile organic compounds (VOCs) is however a key driver in many industrial applications, driven by environmental legislation or cost savings. Where the polymer is used in a solution formulation, improved solubility is also an advantage.

There is therefore a need for high molecular weight polymers possessing low solution or melt viscosities at a viable cost.

That is, there exists the need for polymeric materials which are able to provide solutions or formulations of reduced viscosity but which may be used without the need for vast quantities of volatile organic solvents (VOCs) required to solubilise such compounds. That is, there is a need for a solution or formulation of branched high molecular weight addition copolymers with a lower viscosity than an analogous linear polymeric material. In addition, by reducing the volatile organic solvent (VOC) content and thereby preparing solutions of greater solids content, that is, concentrates, significant savings may also be made during the transportation of formulations containing the polymeric materials.

Additionally a reduction in the melt or solution elasticity behaviour provides a particular advantage over linear polymer analogues of equivalent or greater weight average molecular weight in terms of the ease of application of the formulation via jetting, injection, spraying or roller applying. Additionally, the reduced elastic behaviour can lead to benefits in pumping or processing melt or solutions of branched addition polymers.

It has been found that highly branched dendritic polymers impart a lower solution or melt viscosity when compared to an analogous linear polymer. Dendritic polymers are prepared via a multi-step synthetic route and are limited by chemical functionality and ultimate molecular weight, being prepared at high cost; such dendritic polymers have therefore only found only limited high-cost industrial applications. Branched polymers are typically prepared via a step-growth procedure and again are limited by their chemical functionality and molecular weight although the reduced cost of their manufacture makes branched polymers more attractive from an industrial manufacturing point of view.

Through previous disclosures the inventors of this application have shown that branched polymers of high molecular weight can be prepared via a one-step process using commercially available monomers, such as in WO 2008/071662. Through specific monomer choices the chemical functionality of these polymers can be tuned depending on the specific application. These benefits therefore give advantages over dendritic or step-growth branched polymers. Since branched polymers of high molecular weight may be prepared via an addition process from commercially available monomers, the branched copolymers may be used as a direct replacement for equivalent linear addition polymers and since branched copolymers of high molecular weight comprise a carbon-carbon backbone, such polymers are not susceptible to thermal or hydrolytic instability unlike ester-based dendrimers or step-growth branched polymers. It has also been observed by the inventors that these polymers dissolve faster than equivalent linear polymers.

The branched copolymers of the present invention are branched, non-cross-linked addition polymers and include statistical, block, graft, gradient and alternating branched copolymers. The copolymers of the present invention comprise at least two chains which are covalently linked by a bridge other than at their ends, that is, a sample of said copolymer comprises on average at least two chains which are covalently linked by a bridge other than at their ends. When a sample of the copolymer is made there may be unintentionally some polymer molecules that are un-branched, which is inherent to the production method (addition polymerisation process). For the same reason, a small quantity of the polymer will not have a chain transfer agent (CTA) reside on the chain end. Most importantly however, the branched copolymers described herein according to the present invention possess lower solution or melt viscosities than those of equivalent linear analogues.

There is therefore a need for a form of polymer which can be readily produced at a reasonable cost on an industrial scale and which is able to be used at a reduced solution or melt viscosity in place of linear analogues in various application technologies and systems.

Also, and more importantly, there is a requirement for a form of polymer which can be readily produced at a reasonable cost on an industrial scale and which is able to be used in solution or melt formulations whereby the viscosities of the formulations are lower than those achieved when using equivalent linear polymer analogues.

The applicants have now surprisingly found that branched addition copolymers meet the above criteria. That is, due to the reduced solution or melt viscosity of the branched addition polymers such polymers may be employed in a variety of applications to great effect. For example, when employed in formulations the pumping, spraying and film blowing of the formulations comprising such copolymers is much less arduous leading to cost savings in terms of both energy expedition and time of application.

Therefore according to a first aspect of the present invention there is provided the use of a branched addition copolymer in a solution formulation or melt formulation as a complete replacement for a linear polymer analogue of comparable weight average molecular weight wherein the viscosity of the branched addition copolymer solution and/or melt is lower than the viscosity of the equivalent linear polymer analogue solution formulation and/or melt formulation of at least comparable weight average molecular weight and weight concentration and wherein the branched addition copolymer is obtainable by an addition polymerisation process.

The branched addition copolymer may be of higher weight average molecular weight and weight concentration than the equivalent linear polymer analogue solution formulation and/or melt formulation. Alternatively, the branched addition copolymer may be of an equal weight average molecular weight and weight concentration than the equivalent linear polymer analogue solution formulation and/or melt formulation.

In accordance with the first aspect of the present invention when using the branched addition copolymer, the viscosity of the solution formulation or melt formulation, is at least 90% of the solution formulation or melt formulation of the linear polymer analogue. Alternatively, the viscosity of the solution formulation or melt formulation, is at least 70% of the viscosity of the solution formulation or melt formulation of the linear polymer analogue. Alternatively, the viscosity of the solution formulation or melt formulation, is at least 50% of the viscosity of the solution formulation or melt formulation of the linear polymer analogue. Also as an alternative, the viscosity of the solution formulation or melt formulation, is at least 20% of the viscosity of the solution formulation or melt formulation of the linear polymer analogue.

Likewise, as a further alternative the viscosity of the solution formulation or melt formulation, is between 80 to 10% of the viscosity of the solution formulation or melt formulation of the linear polymer analogue.

It is preferred that in accordance with the first aspect of the present invention the branched addition copolymer comprises a weight average molecular weight of 2,000 Da to 1,500,000 Da. Alternatively, the branched addition copolymer comprises a weight average molecular weight of 2,000 Da to 1,000,000 Da. It is preferred however that the branched addition copolymer comprises a weight average molecular weight of 6,000 Da to 700,000 Da.

Also in accordance with the first aspect of the present invention it is preferred that the branched addition copolymer comprises:
at least two chains which are covalently linked by a bridge other than at their ends; and wherein
the at least two chains comprise at least one ethyleneically monounsaturated monomer, and wherein
the bridge comprises at least one ethylenically polyunsaturated monomer; and wherein
the polymer comprises a residue of a chain transfer agent and/or optionally a residue of an initiator; and wherein
the mole ratio of polyunsaturated monomer(s) to monounsaturated monomer(s) is in a range of from 1:100 to 1:4.

More preferably the branched addition copolymer comprises:
at least two chains which are covalently linked by a bridge other than at their ends; and wherein
the at least two chains comprise at least one ethyleneically monounsaturated monomer, and wherein
the bridge comprises at least one ethylenically polyunsaturated monomer; and wherein
the polymer comprises a residue of a chain transfer agent and/or optionally a residue of an initiator; and wherein
the mole ratio of polyunsaturated monomer(s) to monounsaturated monomer(s) is in a range of from 1:100 to 1:4.
The branched addition copolymers for use according to the first aspect of the present invention are preferably polymerised to greater than 99% conversion.
It is also preferred that the branched copolymer comprises less than 1% monomer impurity.

Furthermore the use of a branched addition copolymer in accordance with the first aspect of the present invention as the replacement in a solution or melt of a linear polymer analogue provides a melt or solution of higher solids content with equivalent viscosity. The solids content of the melt or solution is preferably increased by at least 5%.

The branched addition copolymers can be used according to the first aspect of the present invention to reduce the viscosity of a solution and/or melt in the application areas selected from the group comprising:
coatings, inks, adhesives, lubricants, composites, oil field recovery agents, metal working fluids, coolants, sealants, films, resins, textiles, injection mouldings, water treatment, electronics, cosmetics, pharmaceuticals, agrochemicals, and lithography.

The monomers used to prepare the branched addition copolymers used in accordance with the first aspect of the present invention are vinylic or allylic and are selected from the group comprising: styrenics, acrylics, methacrylics, allylics, acrylamides, methacrylamides, vinyl or allyl acetates, N-vinyl or allyl amines and vinyl or allyl ethers.

Preferred branched addition copolymers according to the first and second aspects of the present invention contain units selected from the groups consisting of: styrene, vinyl benzyl chloride, 2-vinyl pyridine, 4-vinyl pyridine, methyl acrylate, ethyl acrylate, methyl methacrylate, ethyl methacrylate, butyl methacrylate, butyl acrylate, acrylic acid, methacrylic acid, 2-hydroxylethyl methacrylate, 2-hydroxy ethyl acrylate, 2-hydroxypropyl acrylate, 2-hydroxypropyl methacrylate, acrylamide, methacrylamide, dimethyl acrylamide, dimethyl(meth)acrylamide, allyl methacrylate, dimethylaminoethyl methacrylate, dimethylaminoethyl acrylate, diethylaminoethyl methacrylate, diethylaminoethyl acrylate, divinyl benzene, ethyleneglycol dimethacrylate, ethyleneglycol di acrylate, triethylene glycol dimethacrylate, tetraethyleneglycol dimethacrylate, triethyleneglycol diacrylate, tetraethyleneglycol diacrylate, 1,3,5-triallyl-1,3,5-triazine-2,4,6(1H,3H,5H)-trione, dodecane thiol, hexane thiol, 2-mercaptoethanol and fragments arising from azobis isobutyronitrile, di-t-butyl peroxide and t-butyl peroxybenzoate.

More preferably the branched addition copolymers according to the first and second aspects of the present invention comprise units selected from the groups consisting of:
styrene, 2-vinyl pyridine, 4-vinyl pyridine, methyl acrylate, methyl methacrylate, butyl methacrylate, butyl acrylate, acrylic acid, methacrylic acid, acrylamide, methacrylamide, dimethyl acrylamide, dimethyl(meth)acrylamide, divinyl benzene, ethyleneglycol dimethacrylate, ethyleneglycol diacrylate, triethylene glycol dimethacrylate, dodecane thiol, hexane thiol, 2-mercaptoethanol, azobis iso-butyronitrile, di-t-butyl peroxide and t-butyl peroxybenzoate.

Also, in accordance with a second aspect of the present invention there is provided a formulation comprising a branched addition copolymer as described in relation to the first aspect of the present invention and a liquid medium wherein the liquid medium comprises an organic solvent and/or an aqueous solvent and wherein the formulation is used in place of an analogous linear polymer to reduce the solution viscosity of a solution comprising an equivalent linear polymer according to a first aspect of the present invention.

The branched copolymer may also be used as a direct replacement for a linear analogue in a melt formulation with or without the use of a solvent.

The ratio of branched addition copolymer to liquid medium comprises from 1 to 99%.

### Applications

It has been found that in accordance with the first aspect of the present invention the branched addition copolymers may be used for a variety of applications including but not limited to:

i) The use of the branched addition copolymers in coatings, where the low viscosity of the branched addition copolymer can lead to high solids content formulations with lower quantities of volatile organic compounds (VOCs) than the linear equivalent polymers. Additional advantages include faster drying times and ease of application of the formulation. That is higher solids content or lower viscosity coatings can be formulated through the incorporation of the described branched addition copolymers resulting in a formulation which can provide faster drying, easier application and with a reduction in the VOC content of the formulation.

ii) The use of the branched addition copolymers in for example ink formulation. This again leads to the production of lower viscosity inks, with the resultant ability to incorporate a higher ‘payload’ of pigments, dyes or other adjuncts into the ink formulation in addition to easier application of the ink onto substrate.

iii) Use of the branched addition copolymers in adhesives, wherein the use of said branched addition copolymers leads to lower viscosity adhesives with a higher proportion of curable or pressure-sensitive adhesive actives present in the adhesive composition and the possibility for further penetration into the substrate or matrix to be adhered to.

iv) Use of the branched addition copolymers as lubricants, wherein a higher proportion of lubricating branched addition copolymer in a formulation leads to greater friction reducing power with improved viscosity indices. For example, incorporating branched addition copolymers into a lubricant formulation can produce improvements in high temperature lubricity within a lubricant formulation due to the potential to add a large amount of branched copolymer without increasing the low temperature viscosity.

v) Use of the branched addition copolymers as composites, wherein the use of a low viscosity branched addition copolymer in a composite is able to improve the penetration of the polymer into the composite matrix aiding cure levels and increasing the final strength of the polymer.

vi) Use of the branched addition copolymers as oil field recovery agents, where it is a) required to prepare formulations with a higher solids content and b) less deformable recovery agents for oil-field applications with improved, that is, more Newtonian viscosity profiles.

vii) Use of the branched addition copolymers in cutting fluids, wherein the branched addition copolymers provide a means to achieve higher solids content in the cutting fluids with improved friction reduction and heat transfer capabilities.

viii) Use of the branched addition copolymers in coolants, where the use of the branched addition copolymers provides coolant formulations with lower viscosities providing easier transport and pumping characteristics.

ix) Use of the branched addition copolymers in sealant formulations wherein high modulus sealants are prepared and wherein the formulations have the advantages of higher cure rate, lower volatile organic compound (VOC) levels and greater overall strength due to the high solids content. In addition, the branched addition copolymers may be used to produce low viscosity, low volatile organic compounds (VOC) sealant foam formulations.

x) Use of the branched addition copolymers in the production of films wherein low volatile organic compound (VOC) incorporation and high solids content films may be cast using branched copolymers wherein the production of fast film formation is an advantage. In addition melt processing to blow films can be achieved at lower process temperatures.

xi) Use of the branched addition copolymers in resins, wherein efficient solution or melt processing of resins can be achieved as a result of the use of said copolymers. In solution processing the key advantages are the preparation of high solids formulations with low viscosities and lowered incorporation of volatile organic compounds (VOCs). In melt processing, lower production temperatures can also be achieved as a result of the presence of the polymers.

xii) The branched addition copolymers of the present invention may also be used for textile applications where low viscosity, low volatile organic compound levels (VOC), with high solids content for textile treatments comprising the branched addition copolymers is required. This leads to greater textile penetration of the polymer. In melt-spinning techniques, lower temperatures are also achievable by using the branched addition copolymers in the textile formulations.

xiii) The branched addition copolymers may also be used in injection moulding techniques where the use of a branched addition copolymer leads to a reduction in the process times and temperatures required due to the lower melt viscosity.

xiv) The branched addition copolymers may be used in lithography applications wherein the use of the branched addition copolymers with reduced viscosity can be utilised as a resist in lithography, whereby the lower viscosity aids the formation of more precise templates or structures.

xv) The branched addition copolymer described in accordance with the present invention may also be used in place of a linear equivalent polymer in a solution or melt to achieve a solution or melt with reduced elastic behaviour. The branched copolymer materials may then be used at higher solids level in for example a solution formulation, whereby the formulation has a lower elastic profile. As a result such formulations may be sprayed, rolled, injected, mixed, and processed much easier and with less energy input than equivalent linear polymeric materials. This is also true when the polymeric materials are in melt form with the consequence that much lower melt temperatures may be used.

xvi) The branched addition copolymers may also be used in applications where the lower elastic behaviour of the branched polymer formulation results in generally an easier application of a solution or melt of the branched polymer such as by extrusion, injection, jet-application or spraying compared to linear polymers. This can result in faster application times and lower temperatures required for melt applications of higher solids content of the polymer in a solution formulation.

It will be appreciated that in all of the uses described above the branched addition copolymers are used as a replacement for linear polymeric material.

The chain transfer agent (CTA) is a molecule which is known to reduce molecular weight during a free-radical polymerisation via a chain transfer mechanism. These agents may be any thiol-containing molecule and can be either monofunctional or multifunctional. The agent may be hydrophilic, hydrophobic, amphiphilic, anionic, cationic, neutral, zwitterionic or responsive, for example to pH or temperature. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis (borondifluorodimethyl-glyoximate) (CoBF) may also be used. Suitable thiols include but are not limited to: C₂ to C₁₈ branched or linear alkyl thiols such as dodecane thiol, functional thiol compounds such as thioglycolic acid, thio propionic acid, thioglycerol, cysteine and cysteamine. Thiol-containing oligomers or polymers may also be used such as for example poly(cysteine) or an oligomer or polymer which has been post-functionalised to give a thiol group(s), such as poly(ethyleneglycol) (di)thio glycollate, or a pre-formed polymer functionalised with a thiol group. For example, the reaction of an end or side-functionalised alcohol such as poly(propylene glycol) with thiobutyrolactone, will give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerisation including alkyl halides, ally-functional compounds and transition metal salts or complexes. More than one chain transfer agent may be used in combination.

Hydrophobic CTAs include but are not limited to: linear and branched alkyl and aryl (di)thiols such as dodecanethiol, octadecyl mercaptan, 2-methyl-1-butanethiol and 1,9-nonanedithiol. Hydrophobic macro-CTAs (where the molecular weight of the CTA is at least 1000 Daltons) can be prepared from hydrophobic polymers synthesised by RAFT (or MADIX) followed by reduction of the chain end, or alternatively the terminal hydroxyl group of a preformed hydrophobic polymer can be post functionalised with a compound such as thiobutyrolactone. Non-thiol CTA's such as 2,4-diphenyl-4-methyl-1-pentene can also be used.

Hydrophilic CTAs typically contain hydrogen bonding and/or permanent or transient charges. Hydrophilic CTAs include but are not limited to: thio-acids such as thioglycolic acid and cysteine, thioamines such as cysteamine and thio-alcohols such as 2-mercaptoethanol, thioglycerol and ethylene glycol mono- (and di-thio glycollate. Hydrophilic macro-CTAs (where the molecular weight of the CTA is at least 1000 Daltons) can also be prepared from hydrophilic polymers synthesised by RAFT (or MADIX) followed by reduction of the chain end, or alternatively the terminal hydroxyl group of a preformed hydrophilic polymer can be post functionalised with a compound such as thiobutyrolactone.

Amphiphilic CTAs can also be incorporated in the polymerisation mixture, these materials are typically hydrophobic alkyl-containing thiols possessing a hydrophilic function such as but not limited to a carboxylic acid group. Molecules of this type include mercapto undecylenic acid.

Responsive macro-CTAs (where the molecular weight of the CTA is at least 1000 Daltons) can be prepared from responsive polymers synthesised by RAFT (or MADIX) followed by reduction of the chain end, or alternatively the terminal hydroxyl group of a preformed responsive polymer, such as poly(propylene glycol), can be post functionalised with a compound such as thiobutyrolactone.

The residue of the chain transfer agent may comprise 0 to 80 mole % of the copolymer (based on the number of moles of monofunctional monomer). More preferably the residue of the chain transfer agent comprises 0 to 50 mole %, even more preferably 0 to 40 mole % of the copolymer (based on the number of moles of monofunctional monomer). However, most especially the chain transfer agent comprises 0.05 to 30 mole %, of the copolymer (based on the number of moles of monofunctional monomer).

The initiator is a free-radical initiator and can be any molecule known to initiate free-radical polymerisation such as for example azo-containing molecules, persulfates, redox initiators, peroxides, benzyl ketones. These may be activated via thermal, photolytic or chemical means. Examples of these include but are not limited to: 2,2′-azobisisobutyronitrile (AIBN), azobis(4-cyanovaleric acid), benzoyl peroxide, diisopropyl peroxide, t-butyl peroxybenzoate (for example Luperox® P), di-t-butyl peroxide (for example Luperox® DI), cumylperoxide, 1-hydroxycyclohexyl phenyl ketone, hydrogenperoxide/ascorbic acid. lniferters such as benzyl-N,N-diethyldithiocarbamate can also be used. In some cases, more than one initiator may be used. The initiator may be a macroinitiator having a molecular weight of at least 1000 Daltons. In this case, the macroinitiator may be hydrophilic, hydrophobic, or responsive in nature.

Preferably, the residue of the initiator in a free-radical polymerisation comprises from 0 to 10% w/w of the copolymer based on the total weight of the monomers. More preferably the residue of the initiator in a free-radical polymerisation comprises from 0.001 to 8% w/w of the copolymer, and especially 0.001 to 5% w/w, of the copolymer based on the total weight of the monomers.

The use of a chain transfer agent and an initiator is preferred. However, some molecules can perform both functions.

Hydrophilic macroinitiators (where the molecular weight of the pre-formed polymer is at least 1000 Daltons) can be prepared from hydrophilic polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Hydrophobic macroinitiators (where the molecular weight of the preformed polymer is at least 1000 Daltons) can be prepared from hydrophobic polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Responsive macroinitiators (where the molecular weight of the preformed polymer is at least 1000 Daltons) can be prepared from responsive polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

The monofunctional monomer may comprise any carbon-carbon unsaturated compound which can be polymerised by an addition polymerisation mechanism, for example vinyl and allyl compounds. The monofunctional monomer may be hydrophilic, hydrophobic, amphiphilic, anionic, cationic, neutral or zwitterionic in nature. The monofunctional monomer may be selected from but not limited to monomers such as:
vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof.

Other suitable monofunctional monomers include: hydroxyl-containing monomers and monomers which can be post-reacted to form hydroxyl groups, acid-containing or acid-functional monomers, zwitterionic monomers and quaternised amino monomers. Oligomeric, polymeric and di- or multi-functionalised monomers may also be used, especially oligomeric or polymeric (meth)acrylic acid esters such as mono(alkyl/aryl) (meth)acrylic acid esters of polyalkyleneglycol or polydimethylsiloxane or any other mono-vinyl or allyl adduct of a low molecular weight oligomer. Mixtures of more than one monomer may also be used to give statistical, graft, gradient or alternating copolymers.

Vinyl acids and derivatives thereof include: (meth)acrylic acid, fumaric acid, maleic acid, vinyl sulfonic acid, vinyl phosphoric acid, 2-acrylamido-2-methylpropane sulfonic acid, itaconic acid and acid halides thereof such as (meth)acryloyl chloride. Vinyl acid esters and derivatives thereof include: C₁ to C₂₀ alkyl(meth)acrylates (linear and branched) such as for example methyl (meth)acrylate, stearyl (meth)acrylate and 2-ethyl hexyl (meth)acrylate; aryl(meth)acrylates such as for example benzyl (meth)acrylate; tri(alkyloxy)silylalkyl(meth)acrylates such as trimethoxysilylpropyl(meth)acrylate; and activated esters of (meth)acrylic acid such as N-hydroxysuccinamido (meth)acrylate.

Vinyl aryl compounds and derivatives thereof include: styrene, acetoxystyrene, styrene sulfonic acid, 2- and 4-vinyl pyridine, vinyl naphthalene, vinylbenzyl chloride and vinyl benzoic acid.

Vinyl acid anhydrides and derivatives thereof include: maleic anhydride. Vinyl amides and derivatives thereof include: (meth)acrylamide, N-(2-hydroxypropyl)methacrylamide, N-vinyl pyrrolidone, N-vinyl formamide, (meth)acrylamidopropyl trimethyl ammonium chloride, [3-((meth)acrylamido)propyl] dimethyl ammonium chloride, 3-[N-(3-(meth)acrylamidopropyl)-N,N-dimethyl]amino propane sulfonate, methyl (meth)acrylamidoglycolate methyl ether and N-isopropyl(meth)acrylamide.

Vinyl ethers and derivatives thereof include: methyl vinyl ether.

Vinyl amines and derivatives thereof include: dimethylaminoethyl (meth)acrylate, diethylaminoethyl (meth)acrylate, diisopropylaminoethyl (meth)acrylate, mono-t-butylaminoethyl (meth)acrylate, morpholinoethyl(meth)acrylate and monomers which can be post-reacted to form amine groups, such as N-vinyl formamide.

Vinyl aryl amines and derivatives thereof include: vinyl aniline, 2 and 4-vinyl pyridine, N-vinyl carbazole and vinyl imidazole.

Vinyl nitriles and derivatives thereof include: (meth)acrylonitrile.

Vinyl ketones or aldehydes and derivatives thereof include: acrolein.

Hydroxyl-containing monomers include:

vinyl hydroxyl monomers such as hydroxyethyl (meth)acrylate, 1- and 2-hydroxy propyl (meth)acrylate, glycerol mono(meth)acrylate and sugar mono(meth)acrylates such as glucose mono(meth)acrylate,

monomers which can be post-reacted to form hydroxyl groups include: vinyl acetate, acetoxystyrene and glycidyl (meth)acrylate,

acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl (meth)acrylate.

Zwitterionic monomers include: (meth)acryloyl oxyethylphosphoryl choline and betaines, such as [2-((meth)acryloyloxy)ethyl] dimethyl-(3-sulfopropyl)ammonium hydroxide.

Quaternised amino monomers include: (meth)acryloyloxyethyltri-(alk/aryl)ammonium halides such as (meth)acryloyloxyethyltrimethyl ammonium chloride.

Vinyl acetate and derivatives thereof can also be utilised.

Oligomeric and polymeric monomers include: oligomeric and polymeric (meth)acrylic acid esters such as mono(alkyl/aryl)oxypolyalkyleneglycol(meth)acrylates and mono(alkyl/aryl)oxypolydimethyl-siloxane(meth)acrylates. These esters include for example: monomethoxy oligo(ethyleneglycol) mono(meth)acrylate, monomethoxy oligo(propyleneglycol) mono(meth)acrylate, monohydroxy oligo(ethyleneglycol) mono(meth)acrylate, monohydroxy oligo(propyleneglycol) mono(meth)acrylate, monomethoxy poly(ethyleneglycol) mono(meth)acrylate, monomethoxy poly(propyleneglycol) mono(meth)acrylate, monohydroxy poly(ethyleneglycol) mono(meth)acrylate and monohydroxy poly(propyleneglycol) mono(meth)acrylate.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as poly(1,4-butadiene).

The corresponding allyl monomers to those listed above can also be used where appropriate.

Examples of monofunctional monomers include:

amide-containing monomers such as (meth)acrylamide, N-(2-hydroxypropyl)methacrylamide, N,N′-dimethyl(meth)acrylamide, N and/or N′-di(alkyl or aryl) (meth)acrylamide, N-vinyl pyrrolidone, [3-((meth)acrylamido)propyl] trimethyl ammonium chloride, 3-(dimethylamino)propyl(meth)acrylamide, 3-[N-(3-(meth)acrylamidopropyl)-N,N-dimethyl]aminopropane sulfonate, methyl (meth)acrylamidoglycolate methyl ether and N-isopropyl(meth)acrylamide;

(meth)acrylic acid and derivatives thereof such as (meth)acrylic acid, (meth)acryloyl chloride (or any halide), (alkyl/aryl)(meth)acrylate; functionalised oligomeric or polymeric monomers such as monomethoxy oligo(ethyleneglycol) mono(meth)acrylate, monomethoxy oligo(propyleneglycol) mono(meth)acrylate, monohydroxy oligo(ethyleneglycol) mono(meth)acrylate, monohydroxy oligo(propyleneglycol) mono(meth)acrylate, monomethoxy poly(ethyleneglycol) mono(meth)acrylate, monomethoxy poly(propyleneglycol) mono(meth)acrylate, monohydroxy poly(ethyleneglycol) mono(meth)acrylate, monohydroxy poly(propyleneglycol) mono(meth)acrylate, glycerol mono(meth)acrylate and sugar mono(meth)acrylates such as glucose mono(meth)acrylate;

vinyl amines such as aminoethyl (meth)acrylate, dimethylaminoethyl (meth)acrylate, diethylaminoethyl (meth)acrylate, diisopropylaminoethyl (meth)acrylate, mono-t-butylamino (meth)acrylate, morpholinoethyl(meth)acrylate; vinyl aryl amines such as vinyl aniline, vinyl pyridine, N-vinyl carbazole, vinyl imidazole, and monomers which can be post-reacted to form amine groups, such as vinyl formamide;

vinyl aryl monomers such as styrene, vinyl benzyl chloride, vinyl toluene, a-methyl styrene, styrene sulfonic acid, vinyl naphthalene and vinyl benzoic acid;

vinyl hydroxyl monomers such as hydroxyethyl (meth)acrylate, hydroxy propyl (meth)acrylate, glycerol mono(meth)acrylate or monomers which can be post-functionalised into hydroxyl groups such as vinyl acetate, acetoxy styrene and glycidyl (meth)acrylate;

acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, vinyl sulfonic acid, vinyl phosphoric acid, 2-acrylamido-2-methylpropane sulfonic acid, and mono-2-((meth)acryloyloxy)ethyl succinate or acid anhydrides such as maleic anhydride;

zwitterionic monomers such as (meth)acryloyl oxyethylphosphoryl choline and betaine-containing monomers, such as [2-((meth)acryloyloxy)ethyl] dimethyl-(3-sulfopropyl)ammonium hydroxide;

quaternised amino monomers such as (meth)acryloyloxyethyltrimethyl ammonium chloride.

vinyl acetate or vinyl butanoate or derivatives thereof.

The corresponding allyl monomer, where applicable, can also be used in each case.

Functional monomers, that is monomers with reactive pendant groups which can be pre or post-modified with another moiety following polymerisation can also be used such as for example; glycidyl (meth)acrylate, tri(alkoxy)silylalkyl(meth)acrylates such as trimethoxysilylpropyl(meth)acrylate, (meth)acryloyl chloride, maleic anhydride, hydroxyalkyl (meth)acrylates, (meth)acrylic acid, vinylbenzyl chloride, activated esters of (meth)acrylic acid such as N-hydroxysuccinamido (meth)acrylate and acetoxystyrene.

Macromonomers (monomers having a molecular weight of at least 1000 Daltons) are generally formed by linking a polymerisable moiety, such as a vinyl or allyl group, to a pre-formed monofunctional polymer via a suitable linking unit such as an ester, an amide or an ether. Examples of suitable polymers include: mono functional poly(alkylene oxides) such as monomethoxy[poly(ethyleneglycol)] or monomethoxy[poly(propyleneglycol)], silicones such as poly(dimethylsiloxane)s, polymers formed by ring-opening polymerisation such as poly(caprolactone) or poly(caprolactam) or mono-functional polymers formed via living polymerisation such as poly(1,4-butadiene).

Preferred macromonomers include: monomethoxy[poly(ethyleneglycol)] mono(methacrylate), monomethoxy[poly(propyleneglycol)] mono(methacrylate) and mono(meth)acryloxypropyl-terminated poly(dimethylsiloxane).

When the monofunctional monomer is providing the necessary hydrophilicity in the copolymer, it is preferred that the monofunctional monomer is a residue of a hydrophilic monofunctional monomer, preferably having a molecular weight of at least 1000 Daltons.

Hydrophilic monofunctional monomers include: (meth)acryloyl chloride, N-hydroxysuccinamido (meth)acrylate, styrene sulfonic acid, maleic anhydride, (meth)acrylamide, N-(2-hydroxypropyl)methacrylamide, N-vinyl pyrrolidinone, N-vinyl formamide, quaternised amino monomers such as (meth)acrylamidopropyl trimethyl ammonium chloride, [3-((meth)acrylamido)propyl]trimethyl ammonium chloride and (meth)acryloyloxyethyltrimethyl ammonium chloride, 3[N-(3-(meth)acrylamidopropyl)-N,N-dimethyl]aminopropane sulfonate, methyl (meth)acrylamidoglycolate methyl ether, glycerol mono(meth)acrylate, monomethoxy and monohydroxy oligo(ethylene oxide) (meth)acrylate, sugar mono(meth)acrylates such as glucose mono(meth)acrylate, (meth)acrylic acid, vinyl phosphonic acid, fumaric acid, itaconic acid, 2-(meth)acrylamido 2-ethyl propanesulfonic acid, mono-2-((meth)acryloyloxy)ethyl succinate, ammonium sulfatoethyl (meth)acrylate, (meth)acryloyl oxyethylphosphoryl choline and betaine-containing monomers such as [2-((meth)acryloyloxy)ethyl] dimethyl-(3-sulfopropyl)ammonium hydroxide.

Hydrophilic macromonomers may also be used and include: monomethoxy and monohydroxy poly(ethylene oxide) (meth)acrylate and other hydrophilic polymers with terminal functional groups which can be post-functionalised with a polymerisable moiety such as (meth)acrylate, (meth)acrylamide or styrenic groups.

Hydrophobic monofunctional monomers include: C₁ to C₂₈ alkyl (meth)acrylates (linear and branched) and (meth)acrylamides, such as methyl (meth)acrylate and stearyl (meth)acrylate, aryl(meth)acrylates such as benzyl (meth)acrylate, tri(alkyloxy)silylalkyl(meth)acrylates such as trimethoxysilylpropyl(meth)acrylate, styrene, acetoxystyrene, vinylbenzyl chloride, methyl vinyl ether, vinyl formamide, (meth)acrylonitrile, acrolein, 1- and 2-hydroxy propyl (meth)acrylate, vinyl acetate, 5-vinyl 2-norbornene, Isobornyl methacrylate and glycidyl (meth)acrylate.

Hydrophobic macromonomers may also be used and include: monomethoxy and monohydroxy poly(butylene oxide) (meth)acrylate and other hydrophobic polymers with terminal functional groups which can be post-functionalised with a polymerisable moiety such as (meth)acrylate, (meth)acrylamide or styrenic groups.

Responsive monofunctional monomers include: (meth)acrylic acid, 2- and 4-vinyl pyridine, vinyl benzoic acid, N-isopropyl(meth)acrylamide, tertiary amine (meth)acrylates and (meth)acrylamides such as 2-(dimethyl)aminoethyl (meth)acrylate, 2-(diethylamino)ethyl (meth)acrylate, diisopropylaminoethyl (meth)acrylate, mono-t-butylaminoethyl (meth)acrylate and N-morpholinoethyl (meth)acrylate, vinyl aniline, 2- and 4-vinyl pyridine, N-vinyl carbazole, vinyl imidazole, hydroxyethyl (meth)acrylate, hydroxypropyl (meth)acrylate, maleic acid, fumaric acid, itaconic acid and vinyl benzoic acid.

Responsive macromonomers may also be used and include: monomethoxy and monohydroxy poly(propylene oxide) (meth)acrylate and other responsive polymers with terminal functional groups which can be post-functionalised with a polymerisable moiety such as (meth)acrylate, (meth)acrylamide or styrenic groups.

Monomers based on styrene or those containing an aromatic functionality such as styrene, α-methyl styrene, vinyl benzyl chloride, vinyl naphthalene, vinyl benzoic acid, N-vinyl carbazole, 2-, 3- or 4-vinyl pyridine, vinyl aniline, acetoxy styrene, styrene sulfonic acid, vinyl imidazole or derivatives thereof may also be used.

The multifunctional monomer or brancher may comprise a molecule containing at least two vinyl groups which may be polymerised via addition polymerisation. The molecule may be hydrophilic, hydrophobic, amphiphilic, neutral, cationic, zwitterionic, oligomeric or polymeric. Such molecules are often known as cross-linking agents in the art and may be prepared by reacting any di- or multifunctional molecule with a suitably reactive monomer. Examples include: di- or multivinyl esters, di- or multivinyl amides, di- or multivinyl aryl compounds, di- or multivinyl alkyl/aryl ethers. Typically, in the case of oligomeric or polymeric di- or multifunctional branching agents, a linking reaction is used to attach a polymerisable moiety to a di- or multifunctional oligomer or polymer. The brancher may itself have more than one branching point, such as T-shaped divinylic oligomers or polymers. In some cases, more than one multifunctional monomer may be used. When the multifunctional monomer is providing the necessary hydrophilicity in the copolymer, it is preferred that the multifunctional monomer has a molecular weight of at least 1000 Daltons.

The corresponding allyl monomers to those listed above can also be used where appropriate.

Preferred multifunctional monomers or branchers include but are not limited to: divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, poly(ethyleneglycol) di(meth)acrylate and poly(propyleneglycol) di(meth)acrylate; divinyl (meth)acrylamides such as methylene bisacrylamide;

silicone-containing divinyl esters or amides such as (meth)acryloxypropyl-terminated poly(dimethylsiloxane);

divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene).

Macrocrosslinkers or macrobranchers (multifunctional monomers having a molecular weight of at least 1000 Daltons) are generally formed by linking a polymerisable moiety, such as a vinyl or aryl group, to a pre-formed multifunctional polymer via a suitable linking unit such as an ester, an amide or an ether. Examples of suitable polymers include: di-functional poly(alkylene oxides) such as poly(ethyleneglycol) or poly(propyleneglycol), silicones such as poly(dimethylsiloxane)s, polymers formed by ring-opening polymerisation such as poly(caprolactone) or poly(caprolactam) or poly-functional polymers formed via living polymerisation such as poly(1,4-butadiene).

Preferred macrobranchers include:

poly(ethyleneglycol) di(meth)acrylate, poly(propyleneglycol) di(meth)acrylate, methacryloxypropyl-terminated poly(dimethylsiloxane), poly(caprolactone) di(meth)acrylate and poly(caprolactam) di(meth)acrylamide.

Branchers include: methylene bisacrylamide, glycerol di(meth)acrylate, glucose di- and tri(meth)acrylate, oligo(caprolactam) and oligo(caprolactone).

Multi end-functionalised hydrophilic polymers may also be functionalised using a suitable polymerisable moiety such as a (meth)acrylate, (meth)acrylamide or styrenic group.

Further branchers include:

divinyl benzene, (meth)acrylate esters such as ethyleneglycol di(meth)acrylate, propyleneglycol di(meth)acrylate and 1,3-butylene di(meth)acrylate, oligo(ethylene glycol) di(meth)acrylates such as tetraethylene glycol di(meth)acrylate, tetra- or tri(meth)acrylate esters such as pentaerythritol tetra(meth)acrylate, trimethylolpropane tri(meth)acrylate and glucose penta(meth)acrylate. Multi end-functionalised hydrophobic polymers may also be functionalised using a suitable polymerisable moiety such as a (meth)acrylate, (meth)acrylamide or styrenic group.

Multifunctional responsive polymers may also be functionalised using a suitable polymerisable moiety such as a (meth)acrylate, (meth)acrylamide or styrenic group such as poly(propylene oxide) di(meth)acrylate.

Styrenic branchers, or those containing aromatic functionality are particularly preferred including divinyl benzene, divinyl naphthalene, acrylate or methacrylate derivatives of 1,4 or 1,3 or 1,2 derivatives of dihydroxy dimethyl benzene and derivatives thereof.

### EXAMPLES

The present invention will now be explained in more detail by reference to the following non-limiting examples.

In the following examples, copolymers are described using the following nomenclature:

(MonomerG)_{g} (Monomer J)ⱼ (Brancher L)ₗ (Chain Transfer Agent) _{d}

wherein the values in subscript are the molar ratios of each constituent normalised to give the monofunctional monomer values as 100, that is, g+j=100. The degree of branching or branching level is denoted by l and d refers to the molar ratio of the chain transfer agent.
For example:

Methacrylic acid₁₀₀ Ethyleneglycol dimethacrylate₁₅ Dodecane thiol₁₅ would describe a polymer containing at a molar ratio of 100:15:15, methacrylic acid:ethyleneglycol dimethacrylate:dodecane thiol

### Abbreviations:

### Monomers:

AA Acrylic Acid
BMA n-Butyl Methacrylate
EMA Ethyl methacrylate
HPMA 2-Hydroxypropyl methacrylate
IBMA i-butyl methacrylate
IBOMA Isobornyl methacrylate
MMA Methyl methacrylate
St Styrene

### Branchers:

DVB Divinyl benzenes 80%
EGDMA Ethyleneglycol dimethacrylate
TEGDMA Triethyleneglycol dimethacrylate

### CTA

2ME 2-mercaptoethanol
DDT 1-Dodecane thiol
HT Hexanethiol

### Initiators

AIBN Azobis isobutyronitrile
DI Luperox DI (Di-t-butyl peroxide)
P Luperox P (t-butyl peroxybenzoate)

### Solvents

BuOAc Butyl Acetate
AD 40 Exxsol AD40
MEK Butan-2-one
MeOH Methanol
MPA 1-Methoxy-2-propyl acetate
PE 100-120 Petroleum Ether 100-120

All materials were obtained from the Aldrich Chemical Company with the exception of Luperox® DI and P from Arkema Chemical Company, and AD 40 from Exxon Mobil.

### Synthesis and Characterisation

### General Procedure

Into a three-necked round bottom flask fitted in a DrySyn® Vortex Overhead Stirrer system and equipped with a condenser the required monomers and solvent were introduced. The solution was then degassed for 10 minutes by bubbling nitrogen through it. The solution was then heated to the appropriate temperature and stirred at 320 revolutions per minute (rpm). Once the expected temperature had been reached, the initiator was added and the reaction was allowed to commence for at least 4 hours and 30 minutes. When the monomer conversion was found to be greater than 99% (measured by ¹H NMR), the reaction mixture was cooled to room temperature and poured into a vessel. The polymers were characterised by Triple Detection-Size Exclusion Chromatography (TD-SEC).

For example

### BP1

### BMA₁₀₀EGDMA₁₅DDT₁₅

BMA (40 g, 0.281 mol), EGDMA (8.36 g, 42 mmol), DDT (8.54 g, 42 mmol) and petroleum ether (100 to 120° C. fraction) (40 g) were added to a 100 mL flask fitted with an overhead stirred. The solution was degassed for 10 minutes by sparging with nitrogen. The solution was then stirred and heated to 120° C. At reflux, the initiator Luperox® DI (0.710 g, 4.8 mmol) was added. After 18 hours, the reaction mixture was cooled to room temperature. The branched polymer was characterised as follows: Mn 2,200 Da, Mw 340,000 Da, Mw/Mn 138, a 0.414, viscosity 206 mPa.s at 25° C.

### Triple Detection-Size Exclusion Chromatography.

Triple Detection-Size Exclusion Chromatography was performed using a Viscotek instrument and includes a GPC max eluent pump and autosampler, which is coupled to a TDA302 column oven and a multidetector module. The columns used were two ViscoGel HHR-H columns and a guard column with an exclusion limit for polystyrene of 10⁷ g·mol⁻¹.

Tetrahydrofuran (THF) was the mobile phase, and the column oven temperature was set to 35° C., and the flow rate was 1 mL·min⁻¹. The samples were prepared for injection by dissolving 10 mg of polymer in 1.5 mL of HPLC grade THF and then filtered with an Acrodisc® 0.2 μm PTFE membrane. 0.1 mL of this mixture was then injected, and data points were collected for 30 minutes. Omnisec software was used to collect and process the signals transmitted from the detectors to the computer and to calculate the molecular weight.

Polymeric example: Linear and Branched.

A series of linear and branched polymers were synthesised according to the general synthetic procedure described above.

The following commercial, linear, materials were also obtained:
LP1: is a commercially available linear methylmethacylate butylmethyacrylate polymer of molecular weight 50,000
LP2: is a commercially available linear methylmethacylate butylmethyacrylate polymer of molecular weight 200,000
LP6: is Polystyrene (Aldrich Chemical Company)
LP7: is Polyacrylic acid (Acros)
LP14: is a commercially available polymethylmethacrylate

In Table 1 there is provided summary details of the linear and branched polymers prepared for use according to the present invention and details of linear polymers prepared for comparative tests.

TABLE 1
Synthetic procedures for the synthesis
of linear and branched polymers

| | | | Temperature | | |
|---|---|---|---|---|---|
| | | | (degrees | | |
| Example | Solid | | centigrade | | Amount of |
| number | content^{a} | Solvent | ° C.) | Initiator | initiator^{b} |
| BP1 | 60 | PE 100-120 | 120 | DI | 2.00% |
| BP2 | 50 | xylene | 145 | DI | 2.00% |
| BP3 | 55 | xylene | 145 | DI | 1.20% |
| BP4 | 55 | xylene | 145 | DI | 1.25% |
| BP5 | 50 | xylene | 70 | AIBN | 2.00% |
| BP6 | 50 | xylene | 145 | DI | 1.20% |
| BP7 | 50 | xylene | 145 | DI | 1.20% |
| BP8 | 50 | xylene | 145 | DI | 1.50% |
| BP9 | 55 | xylene | 145 | DI | 1.50% |
| BP10 | 30 | xylene | 145 | DI | 1.50% |
| BP11 | 35 | MPA | 145 | DI | 1.50% |
| BP12 | 40 | MPA | 145 | DI | 1.50% |
| BP13 | 35 | MPA | 145 | DI | 1.50% |
| BP14 | 42 | MeOH | 70 | AIBN | 0.55% |
| BP15 | 35 | BuOAc | 127 | P | 1.875% |
| BP16 | 35 | BuOAc | 127 | P | 1.875% |
| BP17 | 75 | D40 | 150 | DI | 1.33% |
| BP18 | 75 | D40 | 150 | DI | 1.33% |
| BP19 | 75 | D40 | 150 | DI | 1.33% |
| BP20 | 75 | D40 | 150 | DI | 1.33% |
| BP21 | 55 | xylene | 145 | DI | 2.0% |
| BP22 | 50 | xylene | 145 | DI | 1.5% |
| BP23 | 50 | Xylene | 145 | DI | 1.5% |
| LP3 | 40 | xylene | 145 | DI | 1.50% |
| LP4 | 40 | xylene | 145 | DI | 1.50% |
| LP8 | 30 | BuOAc | 127 | P | 2.25% |
| LP9 | 30 | BuOAc | 127 | P | 2.25% |
| LP10 | 30 | xylene | 145 | DI | 1.50% |
| LP11 | 30 | xylene | 145 | DI | 1.50% |
| LP12 | 30 | xylene | 145 | DI | 1.50% |

In Table 1 above which relates to the details of the linear and branched copolymers synthesised:
^{a}represents the solid content in weight percent (wt. %);
^{b}represents the mole percentage relative to double bonds (Mol. %);
^{c}represents the total time of synthesis.

In Table 2 there is provided the compositional and analytical data recorded for the linear and branched copolymers.

TABLE 2
Compositional and analytical data.

| Example | | | | Mw/ | |
|---|---|---|---|---|---|
| number | Composition^{d} | Mn^{e} | Mw^{e} | Mn | α |
| BP1 | BMA₁₀₀EGDMA₁₅DDT₁₅ | 2.2 | 304 | 138.0 | 0.414 |
| BP2 | MMA₆₈BMA₂₈AA₄DVB₁₅DDT₁₅ | 3.0 | 78 | 26.0 | 0.387 |
| BP3 | MMA₆₈BMA₂₈AA₄DVB₁₅DDT₁₅ | 10.6 | 230 | 22.0 | 0.370 |
| BP4 | MMA₅₈BMA₃₈AA₄DVB₁₅DDT₁₅ | 3.8 | 128 | 34.0 | 0.397 |
| BP5 | MMA₄₈BMA₄₈AA₄DVB₁₅DDT₁₅ | 12.3 | 77 | 6.3 | 0.388 |
| BP6 | MMA₇₈BMA₂₂DVB₁₅ 2ME₁₅ | 5.2 | 151 | 29.0 | 0.437 |
| BP7 | MMA₅₀IBOMA₃₅BMA₁₀AA₅DVB₁₅DDT₁₅ | 1.8 | 43 | 24.0 | 0.523 |
| BP8 | MMA₉₈AA₂EGDMA₁₅DDT₁₆ | 3.3 | 306 | 92.0 | 0.601 |
| BP9 | MMA₉₈AA₂EGDMA₅DDT_{6.5} | 30.0 | 622 | 21.0 | 0.533 |
| BP10 | MMA₉₈AA₂EGDMA₁₅DDT₁₆ | 0.9 | 17 | 19.0 | 0.601 |
| BP11 | ST₁₀₀TEGDMA₅DDT_{5.5} | 60.0 | 790 | 13.0 | 0.540 |
| BP12 | ST₁₀₀TEGDMA₁₀DDT₁₅ | 4.7 | 325 | 69.0 | 0.420 |
| BP13 | ST₁₀₀TEGDMA₁₅DDT_{16.5} | 15.7 | 531 | 34.0 | 0.460 |
| BP14 | AA₁₀₀EGDMA₁₀ 2ME₁₅ | 16.9 | 20.3 | 1.2 | 0.560 |
| BP15 | MMA₄₀BMA₂₀HPMA₄₀EGDMA_{1.33} 2ME₂ | 6.6 | 76.8 | 12.0 | 0.497 |
| BP16 | MMA₄₉BMA_{24.5}HPMA_{26.5}EGDMA_{1.23} | 8.15 | 67.5 | 8.3 | 0.522 |
| | 2ME_{1.85} | | | | |
| BP17 | IBMA₉₅AA₅DVB₂₅DDT₂₈ | 4.4 | 56 | 13.0 | 0.367 |
| BP18 | IBMA₉₅AA₅DVB₃₀DDT₃₀ | 2.7 | 143 | 53.0 | 0.391 |
| BP19 | EMA₉₅AA₅DVB₂₅HT₂₈ | 3.0 | 64 | 21.0 | 0.558 |
| BP20 | EMA₉₅AA₅DVB₃₀DDT₃₀ | 6.2 | 498 | 80.0 | 0.410 |
| BP21 | MMA₅₈BMA₃₈AA₄DVB₁₅DDT₁₅ | 7.8 | 189 | 24 | 0.58 |
| BP22 | MMA₉₈AA₂EGDMA₅DDT_{6.5} | 2.5 | 116 | 46 | 0.415 |
| BP23 | MMA₁₀₀DVB₅ 2ME₈ | 2.0 | 22 | 15 | 0.605 |
| LP1 | MMABMA | N/D | 50 | N/D | N/D |
| LP2 | MMABMA | N/D | 200 | N/D | N/D |
| LP3 | MMA₉₈AA₂DDT₂ | 2.3 | 9.3 | 4.0 | 0.570 |
| LP4 | MMA₉₈AA₂ | 10.0 | 102 | 10.0 | 0.694 |
| LP5 | BMA₁₀₀ | N/D | 320 | N/D | N/D |
| LP6 | ST₁₀₀ | N/D | 192 | N/D | N/D |
| LP7 | AA₁₀₀ | N/D | 5 | N/D | N/D |
| LP8 | MMA₅₀BMA_{23.1}HPMA_{26.9} | 3.4 | 43 | 13.0 | 0.802 |
| LP9 | MMA₄₀BMA_{19.4}HPMA_{40.6} | 3.6 | 46 | 13.0 | 0.780 |
| LP10 | IBMA₉₅AA₅ | 1.0 | 42.6 | 43 | 0.833 |
| LP11 | EMA₉₅AA₅ | 1.2 | 52.3 | 44 | 0.766 |
| LP12 | EMA₉₅AA₅DDT_{0.5} | 0.6 | 17 | 28 | 0.791 |
| LP13 | MMA₅₀BMA₅₀DTT₂ | 5.3 | 10.9 | 2 | 0.63 |
| LP14 | MMA₁₀₀ | N/D | 125 | N/D | N/D |

In Table 2 above which details the compositional and analytical data for the linear and branched copolymers synthesised:
^{d}represents the molar composition of the polymers
^{e}the relative quantities in kg/mol;
Mn represents the number average molecular weight in kg/mol;
Mw/Mn represents the polydispersity of the polymers
α—represents the Mark-Houwink alpha value.
N/D—Not determined

### Viscosity Measurements.

The solution and melt viscosities of the branched addition copolymers were measured as follows.

### Solution:

The branched copolymers were dissolved in an appropriate solvent and made up to a stated percentage weight/weight solutions and the viscosities of the copolymers were measured on a Brookfield DV-II + Pro Viscometer, fitted with a CP-40 or CP-52 at 25 or 60° C. The results are provided in Tables 3 and 4.

### Melt:

The melt viscosity of all of the branched copolymers were measured using a Bohlin CVO 120 controlled stress rheometer fitted with a CP4°/ 40 mm cone. The temperature was set to a pre-determined temperature and the viscosity of the polymer was recorded with increasing shear rate.

In Table 3 there is provided the results of the linear and branched copolymer solution viscosity measurements as tested in xylene at 25° C.

TABLE 3
Solution viscosities of linear and branched
polymer solutions in xylene

| CONCEN- | | | | | | |
|---|---|---|---|---|---|---|
| TRATION | VISCOSITY (mPa · s) | | | | | |
| (%) | LP1 | LP2 | LP5 | BP1 | BP2 | BP3 |
| 20 | 22.8 | 90.0 | 51.2 | 3.0 | 4.9 | ND |
| 30 | 98.1 | 636.0 | 198.7 | 9.2 | 6.5 | 14.0 |
| 40 | 590.0 | 6185.0 | 873.0 | 26.2 | 18.0 | 62.0 |
| 50 | 5650.0 | ND | 7391.0 | 98.0 | 49.0 | 269.0 |
| 70 | ND | ND | ND | 1969.0 | 3980.0 | ND |
| 80 | ND | ND | ND | 4119.0 | 47731.0 | ND |

ND—Not determined

Table 3 indicates that the branched methacrylate-based polymers BP1-3 have lower solution viscosities at a ‘set solids content’ than the linear equivalent polymers with larger weight average molecular weights. This is further highlighted in the case of BP1 and LP1 whose weight average molecular weights are 304,000 and 54,000 Daltons respectively. Where the solids content was equivalent for the linear and branched solutions it is apparent that the branched materials showed much reduced solution viscosities.

In Table 4 there is provided the solution and viscosity measurements for the branched and linear polymers as measured in a range of solvents; xylene, toluene, water, butyl acetate, butan-2-one, AD-40 and petroleum ether.

TABLE 4

| | | | Temperature | |
|---|---|---|---|---|
| | | Concentration | Degrees | |
| Example | | (weight | centigrade | Viscosity |
| number | Solvent | percent wt. %) | ° C. | (mPa · s) |
| BP1 | PE 100-120 | 60 | 25 | 206.0 |
| BP6 | xylene | 50 | 25 | 360.0 |
| BP8 | xylene | 50 | 25 | 123.0 |
| BP8 | toluene | 35 | 25 | 14.3 |
| BP9 | toluene | 35 | 25 | 69.6 |
| BP11 | toluene | 30 | 25 | 32.3 |
| BP12 | toluene | 30 | 25 | 8.0 |
| BP13 | toluene | 30 | 25 | 13.7 |
| BP14 | water | 40 | 25 | 47.6 |
| BP15 | BuOAc | 40 | 25 | 489.0 |
| BP16 | BuOAc | 40 | 25 | 241.0 |
| BP16 | MEK | 50 | 25 | 230.0 |
| BP17 | AD40 | 60 | 25 | 248.0 |
| BP17 | AD40 | 75 | 25 | 2343.0 |
| BP18 | AD40 | 50 | 25 | 154.0 |
| BP18 | AD40 | 70 | 25 | 2319.0 |
| BP19 | AD40 | 60 | 25 | 1148.0 |
| BP19 | AD40 | 70 | 25 | 3859.0 |
| BP20 | AD40 | 50 | 25 | 567.0 |
| BP20 | AD40 | 60 | 25 | 2706.0 |
| LP4 | toluene | 35 | 25 | 1328.0 |
| LP6 | toluene | 30 | 25 | 693 |
| LP7 | water | 40 | 25 | 64.3 |
| LP8 | BuOAc | 40 | 25 | 791.0 |
| LP8 | MEK | 50 | 25 | 462.0 |
| LP9 | BuOAc | 40 | 25 | 787.0 |
| LP10 | AD40 | 50 | 25 | NS |
| LP10 | AD40 | 50 | 60 | 3130.0 |
| LP11 | AD40 | 50 | 60 | NS |
| LP12 | AD40 | 50 | 60 | NS |

NS—Not soluble

In Table 4 the data clearly shows that the branched polymer solutions exhibit lower solution viscosities than their linear polymer equivalents of similar or higher molecular weight in varying solvents. Additionally the branched polymers can be more soluble at a lower solution temperature than the linear equivalents such as in the case of LP 10 and BP 17 where the branched material has a lower solution viscosity at a lower temperature and higher solids content than the linear equivalent.

In Table 5 there is provided the results for the melt viscosities of linear and branched polymers accordingly.

TABLE 5

| | Temperature | | | |
|---|---|---|---|---|
| | (degrees | | | |
| Example | centigrade | VISCOSITY (Pa · s) | | |
| number | ° C.) | 10 s⁻¹ | 50 s⁻¹ | 100 s⁻¹ |
| BP4 | 180 | 0.6 | 0.5 | 0.5 |
| BP5 | 180 | 0.6 | 0.5 | 0.5 |
| BP8 | 160 | 1.9 | 1.5 | 1.5 |
| BP10 | 160 | 0.3 | 0.3 | 0.3 |
| BP17 | 160 | 0.2 | 0.2 | 0.2 |
| BP18 | 160 | 0.4 | 0.4 | 0.4 |
| BP19 | 180 | 0.2 | 0.2 | 0.2 |
| BP20 | 180 | 0.4 | 0.4 | 0.4 |
| BP-21 | 180 | 0.4 | 0.4 | 0.4 |
| LP1 | 180 | 454.0 | ND | ND |
| LP3 | 160 | 57.0 | ND | 53.0 |
| LP10 | 160 | 98.4 | 65.1 | 43.7 |
| LP11 | 180 | 123.7 | 24.1 | 63.4 |
| LP12 | 160 | 42.7 | ND | 35.2 |

ND—Not determined

In Table 5 it can be seen that the branched polymer analogues exhibit lower melt viscosities that their linear equivalent materials. In all cases the linear materials had a smaller weight average molecular weight value than the branched copolymer materials.

### Extensional Rheology

A capillary break up extensional Rheometer (CaBER 1, Thermo Haake) was employed to perform capillary break-up experiments. For each measurement the sample was loaded between the 4 mm plate using a syringe. A fluid circulator bath was used to control of the temperature of the plate at 25° C. The initial sample aspect ratio Λ₀=h₀/2R₀, of 0.5 was defined by initial gap, h₀, of 2 mm and plates diameter, 2R₀ , of 4 cm which compensate the low surface tension. All measurements (Table 6) were performed in triplicate with a strike time of 25 ms and a Hencky strain of 1.14 or 1.15. After the step strain was applied, the decay of filament was monitored by a near infra-red laser diode (Omron ZLA-4) with resolution of 10 μm. The Trotan ratio was calculated by dividing the extensional viscosity by the zero shear viscosity of the polymer solution; since the polymer solutions were Newtonian the zero shear viscosities were taken as an average viscosity over a stain rate of 1 -100 s⁻¹.

### Rotational Rheology

The solution viscosity measurements were performed using an AR2000 cone and plate controlled stress rheometer fitted with a 60 mm 2° anodised cone. Viscosity was measured with increasing shear rate from 1 to 100 s⁻¹ at 25° C. The oscillation rheology of LP14 and BP23 was measured on a Bohlin CVO controlled stress cone and plate rheometer fitted with a CP 2°/55 mm cone.

Surface tension

A torsion balance fitted with a KrUss DuNouy ring was used to measure the surface tension of the liquids at room temperature (25° C.). The extensional rheology results for the polymer solutions are given in Table 6.

TABLE 7

| | Phase | Shear | | | Maximum | Minimum | | | |
|---|---|---|---|---|---|---|---|---|---|
| | angle | Stress | Strain | Frequency | Strain | strain | Initial | Period | Delay |
| Polymer | ° | (Pa) | (N) | (Hz) | (N) | (N) | Stress | (s) | (s) |
| BP23 | 77.00 | 701 | 0.099 | 30 | 1 | 0.0005 | 0.5 | 1024 | 5 |
| LP14 | 67.26 | 845 | 0.266 | 10 | 0.5 | 0.005 | 0.5 | 1024 | 5 |

TABLE 6

| | | | Surface | | Extensional | | | | |
|---|---|---|---|---|---|---|---|---|---|
| Polymer/ | | Density/ | tension/ | Relaxation | Viscosity/ | Experimental | Concentration | Viscosity/ | Trotan |
| blend | Solvent | (g/mL) | (mN/m) | time/s | (mPa · s) | break-up time | (%) | (mPa · s) | ratio |
| LP4 | MEK | 1.05 | 24.7 | 0.0249 | 1.071 | 0.120 | 26.9 | 154 | 7.0 |
| BP22 | MEK | 0.92 | 27.0 | 0.0062 | 0.460 | 0.026 | 50.0 | 114 | 4.0 |
| BP8 | MEK | 0.93 | 25.7 | none | 0.300 | 0.010 | 50.0 | 442 | 2.0 |

TABLE 8

| | | | | Complex | | | | | | Phase | Viscous | Elastic | Complex |
|---|---|---|---|---|---|---|---|---|---|---|---|---|---|
| | Concn. | | Freq. | Viscosity/ | Torque/ | | Temp/ | Shear | Strain/ | Angle/ | modulus/ | modulus/ | Modulus/ |
| Polymer | % w/w | Solvent | Hz | Pas | Nm | Time/s | ° C. | stress | N | ° | MPa | MPa | MPa |
| BP23 | 60 | MPA | 50 | 66.0 | 0.34 | 162 | 25 | 2037 | 0.101 | 74.89 | 20 | 5.4 | 20 |
| BP23 | 60 | MPA | 100 | 47.5 | 0.62 | 338 | 25 | 3695 | 0.099 | 73.95 | 29 | 8.2 | 30 |
| LP14 | 40 | MPA | 50 | 57.0 | 0.023 | 368 | 25 | 1354 | 0.101 | 44.71 | 12 | 12.7 | 18 |
| LP14 | 40 | MPA | 75 | 43.3 | 0.023 | 397 | 25 | 1377 | 0.101 | 41.14 | 13 | 15.4 | 20 |

Concn.—means concentration as a % weight/weight
Freq.—means frequency

1. A method for reducing an elastic behavior of a polymeric solution formulation and/or melt formulation, the method comprising:
adding a branched addition copolymer to the polymeric solution formulation and/or melt formulation as a complete replacement for a linear polymer analogue of comparable weight average molecular weight, resulting in a branched addition copolymer solution formulation and/or melt formulation, wherein viscosity of the branched addition copolymer solution formulation and/or melt formulation is lower than the viscosity of an equivalent linear polymer analogue solution formulation or melt formulation of at least comparable weight average molecular weight and weight concentration, and wherein the branched addition copolymer is obtainable by an addition polymerization process.

2. A method as defined in claim 1, wherein the branched addition copolymer is of at least one of a higher weight average molecular weight and a higher weight concentration than the equivalent linear polymer analogue solution formulation or melt formulation.

3. A method as defined in claim 1, wherein the branched addition copolymer is of at least one of an equal weight average molecular weight and an equal weight concentration to the equivalent linear polymer analogue solution formulation or melt formulation.

4. A method as defined in claim 1, wherein the viscosity is at least 90% of the viscosity of the solution formulation or melt formulation of the linear polymer analogue.

5. A method as defined in claim 1, wherein the viscosity is at least 70% of the viscosity of the solution formulation or melt formulation of the linear polymer analogue.

6. A method as defined in claim 1, wherein the viscosity is at least 50% of the viscosity of the solution formulation or melt formulation of the linear polymer analogue.

7. A method as defined in claim 1, wherein the viscosity is at least 20% of the viscosity of the solution formulation or melt formulation of the linear polymer analogue.

8. A method as defined in claim 1, wherein the viscosity is between 10 to 80% of the viscosity of the solution formulation or melt formulation of the linear polymer analogue.

9. A method as defined in claim 1, wherein the branched addition copolymer comprises a weight average molecular weight of 2,000 Da to 1,500,000 Da.

10. A method as defined in claim 1, wherein the branched addition copolymer comprises a weight average molecular weight of 2,000 Da to 1,000,000 Da.

11. A method as defined in claim 1, wherein the branched addition copolymer comprises a weight average molecular weight of 6,000 Da to 700,000 Da.

12. A method as defined in claim 1, wherein the branched addition copolymer comprises at least two chains which are covalently linked by a bridge other than at their ends, and
wherein the at least two chains comprise at least one ethyleneically monounsaturated monomer,
wherein the bridge comprises at least one ethylenically polyunsaturated monomer, and
wherein the polymer comprises at least one of a residue of a chain transfer agent and a residue of an initiator, and
wherein a mole ratio of polyunsaturated monomers to monounsaturated monomers is in a range of from 1:100 to 1:4.

13. A method as defined in claim 1, wherein the branched addition copolymer comprises at least two chains which are covalently linked by a bridge other than at their ends,
wherein the at least two chains comprise at least one ethyleneically monounsaturated monomer,
wherein the bridge comprises at least one ethylenically polyunsaturated monomer,
wherein the polymer comprises at least one of a residue of a chain transfer agent and a residue of an initiator,
wherein at least one of the monounsaturated monomers and polyunsaturated monomers and chain transfer agents is a hydrophilic residue,
wherein at least one of one of the monounsaturated monomers and polyunsaturated monomers and chain transfer agents is a hydrophobic residue, and
wherein a mole ratio of polyunsaturated monomers to monounsaturated monomers is in a range of from 1:100 to 1:4.

14. A method as defined in claim 1, wherein the branched copolymer comprises less than 1% impurity.

15. A method as defined in claim 1, wherein the replacement of the polymeric solution formulation and/or melt formulation of a linear polymer analogue with the branched addition copolymer provides melt the branched addition copolymer solution formulation and/or melt formulation having a higher solids content with equivalent viscosity.

16. A method as defined in claim 15, wherein the solids content of the melt or solution is increased by at least 5%.

17. A method as defined in claim 1 to reduce the viscosity of the polymeric solution formulation or melt formulation in the application areas selected from the group consisting of:
coatings, inks, adhesives, lubricants, composites, oil field recovery agents, metal working fluids, coolants, sealants, films, resins, textiles, injection mouldings, water treatment, electronics, cosmetics, pharmaceuticals, agrochemicals, and lithography.

18. A method as defined in claim 12, wherein the monomers are vinylic or allylic in nature and are selected from the group comprising:
styrenics, acrylics, methacrylics, allylics, acrylamides, methacrylamides, vinyl or allyl acetates, N-vinyl or allyl amines and vinyl or allyl ethers.

19. A method as defined in claim 1, wherein the branched addition copolymer comprises units are selected from the group consisting of:
styrene, vinyl benzyl chloride, 2-vinyl pyridine, 4-vinyl pyridine, methyl acrylate, ethyl acrylate, methyl methacrylate, ethyl methacrylate, butyl methacrylate, butyl acrylate, acrylic acid, methacrylic acid, 2-hydroxylethyl methacrylate, 2-hydroxy ethyl acrylate, 2-hydroxypropyl acrylate, 2-hydroxypropyl methacrylate, acrylamide, methacrylamide, dimethyl acrylamide, dimethyl(meth)acrylamide, allyl methacrylate, dimethylaminoethyl methacrylate, dimethylaminoethyl acrylate, diethylaminoethyl methacrylate, diethylaminoethyl acrylate, divinyl benzene, ethyleneglycol dimethacrylate, ethyleneglycol di acrylate, triethylene glycol dimethacrylate, tetraethyleneglycol dimethacrylate, triethyleneglycol diacrylate, tetraethyleneglycol diacrylate, 1,3,5-triallyl-1,3,5-triazine-2,4,6(1H,3H,5H)-trione, dodecane thiol, hexane thiol, 2-mercaptoethanol and fragments arising from azobis isobutyronitrile, di-t-butyl peroxide and t-butyl peroxybenzoate.

20. A method as defined in claim 1, wherein the branched addition copolymer comprises units are selected from the group consisting of:
styrene, 2-vinyl pyridine, 4-vinyl pyridine, methyl acrylate, methyl methacrylate, butyl methacrylate, butyl acrylate, acrylic acid, methacrylic acid, acrylamide, methacrylamide, dimethyl acrylamide, dimethyl(meth)acrylamide, divinyl benzene, ethyleneglycol dimethacrylate, ethyleneglycol diacrylate, triethylene glycol dimethacrylate, dodecane thiol, hexane thiol, 2-mercaptoethanol, azobis isobutyronitrile, di-t-butyl peroxide and t-butyl peroxybenzoate.

21. A formulation, comprising:
a branched addition copolymer as defined in claim 1; and
a liquid medium, wherein the liquid medium comprises at least one of an organic solvent and an aqueous solvent for reducing the viscosity of the polymeric solution formulation and/or melt formulation as defined in claim 1.

22. A formulation as defined in claim 21, wherein a ratio of the branched addition copolymer to the liquid medium is from 1 to 99%.
